(19) United States
(12) Patent Application Publication
(10) Pub. No.: US 2005/0079099 A1
(43) Pub. Date: Apr. 14, 2005

(54) Generation of biochemical images and methods of use

(75) Inventors: Michael Spain, Austin, TX (US); Craig Benson, Austin, TX (US); Mark Chandler, Austin, TX (US); James Mapes, Austin, TX (US); Ralph McDade, Austin, TX (US)

Correspondence Address: KATTEN MUCHIN ZAVIS ROSENMAN, 525 WEST MONROE STREET, CHICAGO, IL, 60661-3693, US

(73) Assignee: Rules-Based Medicine, Inc., Austin, TX (US)

(21) Appl. No.: 10/961,145

(22) Filed: Oct. 12, 2004

Related U.S. Application Data
(60) Provisional application No. 60/510,093, filed on Oct. 10, 2003.

(51) Int. Cl. G01N 33/48

(52) U.S. Cl. 422/72; 436/63

(57) ABSTRACT

A biochemical image is derived from specimens, preferably obtained from a statistically relevant sample of a subject population sharing a common characteristic. The biochemical images for one or more conditions present in the subject population and/or one or more test subjects are determined and made available for computer implemented diagnostic evaluations and medical research. In addition to using the information for general research purposes, information from the images may be used to classify diseases and disease states.

### PRIORITY INFORMATION

This application claims priority to U.S. provisional application No. 60/510,093, the disclosure of which is incorporated in its entirety herein.

### FIELD OF THE INVENTION

The present invention relates to methods for profiling and diagnosing various diseases. More particularly, the present invention relates to the generation and use of a biochemical image comprising biochemical data for a wide range of applications, including modeling and study of diseases, diagnosis and prognosis of disease states, and pharmaceutical target identification.

### BACKGROUND OF THE INVENTION

At present, it is known that many diseases at least, in one aspect, manifest changes in homeostatic levels of biochemical analytes present in blood. For example, prostate serum antigen (PSA) levels are generally elevated in patients suffering from prostate cancer and continue to rise as the disease progresses. Similarly, insulin levels are lower in patients diagnosed with diabetes mellitus (Type 1 diabetes).

These determinant analytes are often developed for individual diagnostic tests that are then used to monitor the presence and progression of their respective disease. However, single determinant analytes for many diseases remain unknown or perform poorly in current diagnostic methods.

It is desirable, therefore, in such instances, to analyze a plurality of analytes in profiling and diagnosing a disease. It is further desirable to provide a method of diagnosing a disease based on a single test, comprising a plurality of analytes that, in combination, provide an analyte profile of the respective disease. It is further still desirable to provide a single test that may compile the measurements of a plurality of analytes into a representative biochemical image of the disease. It is yet further desirable to generate a repository of biochemical images of a plurality of diseases, whereby a single test may be used to diagnose a plurality of diseases.

### SUMMARY OF THE INVENTION

In one embodiment, the present invention is directed to a method for generating a biochemical image of a disease comprising: (a) obtaining one or more specimens for a disease from a sample of population of subjects with the disease, (b) assaying each of the specimens for the concentration values of a plurality of biochemical analytes, (c) determining for each disease from the assaying in (b) a distribution of values for each biochemical analyte, and/or comparing distribution values of each analyte with all analytes and/or all analytes with all analytes (d) calculating average values for each of the distribution of values in (c), (e) storing the distribution and average values in a database, and (f) generating a biochemical image representing the values from (d). The methods of the present invention may also include determination of patterns of distribution values among and between analytes. The specimens may be derived from normal subjects or from abnormal subjects comprising any disease characterized by, such as, neoplasia, neurodegeneration, or immunodeficiency. Assays of the method may also comprise microspheres analyzed by flow cytometry.

In accordance with another embodiment of the present invention, A method for identifying a genotype from a biochemical phenotype is provided, comprising: (a) providing one or more test specimens from a subset of a population of subjects with shared a shared genotype, (b) assaying for each of the test specimens for the concentration values of a plurality of biochemical analytes, (c) determining for each genotype from the assaying in (b) a distribution of values for each biochemical analyte, and/or comparing distribution values of each analyte with all analytes and/or all analytes with all analytes, (d) calculating average values for each of the distribution of values in (c), (e) deriving for each genotype from the assaying in (b) a mathematical correlation between the biochemical phenotype obtained from the values calculated in (d) and the genotype, (f) generating a biochemical image comprising the correlation data, (g) providing to the user access to said average values and correlation data in said database, and wherein the number of specimens in (a) includes a sufficient number of specimens such that the values correspond to a statistically significant representation of those values for the population as a whole.

In accordance with another embodiment of the present invention, a method for identifying a disease from a biochemical phenotype is provided comprising: (a) providing one or more test samples derived from a test subject; (b) exposing the one or more test samples to a panel of biochemical assays to gather values for a plurality of biochemical analytes; (c) generating a biochemical image representing the values of (b); (d) comparing the biochemical analyte image generated from the one or more test samples from the test subject with a database of accumulated biochemical analyte image from test samples taken from a plurality of diseases, which accumulated data provides a relationship between one or more predetermined biochemical images and the disease of a plurality of subjects whose accumulated biochemical analyte data share similar features; and (e) identifying a disease in the test subject based, at least in part, on the results of the comparison.

In accordance with yet another embodiment of the present invention, a method of generating an animal model of a disease from a biochemical phenotype of the disease is provided, comprising: (c) obtaining one or more test specimens from a population of subjects with a shared disease; (b) exposing the one or more test samples to a plurality of biochemical assays to gather values for a plurality of biochemical analyte data; (c) determining a relationship between one or more biochemical analyte data images and the disease of the population of subjects whose accumulated biochemical indices share similar features; and (d) genetically manipulating an animal having to comprise one or more biochemical analyte data image associated with the disease of the population of subjects.

In accordance with still yet another embodiment of the present invention, a method of generating an animal model of a genotype from a biochemical phenotype of the disease is provided, comprising: (c) obtaining one or more test specimens from a population of subjects with a shared genotype; (b) exposing the one or more test samples to a plurality of biochemical assays to gather values for a plurality of biochemical analyte data; (c) generating a biochemical image representing the values from (b); (d) determining a relationship between one or more biochemical analyte data and the genotype of the population of subjects whose accumulated biochemical analyte data share similar features; and (e) genetically manipulating an animal having one or more biochemical indices associated with the genotype of the population of subjects.

In accordance with still yet another embodiment of the present invention, a computer implemented method for providing information on a disease to a user is provided, comprising: (a) obtaining one or more test specimens for a plurality of diseases from a subset of a population of subjects with a shared disease, (b) assaying each of the test specimens for the concentration values of a plurality of biochemical analytes, (c) determining for each disease from the assaying in (b) a distribution of values for each biochemical analyte, (d) calculating average values for each of the distribution of values in (c), (e) generating a biochemical image representing the values from (d), and (f) providing to the user access to said distributing and average values in said database.

There has thus been outlined certain embodiments of the invention in order that the detailed description herein may be better understood, and in order that the present contribution to the art may be better appreciated. There are, of course, additional embodiments of the invention which also form the subject matter of the claims appended hereto.

As such, those skilled in the art will appreciate from the disclosure herein that the conception upon which this disclosure is based may readily be utilized as a basis for the designing of other structures, methods and systems in accordance with the present invention. Therefore, the claims should be regarded as including equivalent constructions which may not be explicitly described herein.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

FIG. 1 is an exemplary biochemical analyte data image of the present invention.

FIG. 2 is a flow diagram of a method for imaging a disease. In the method shown, each sample imaged is obtained from a sample consisting of a subset of a population of subjects with shared characteristics, and used to generate a biochemical analyte data image that corresponds to a representation of characteristics of a disease associated with such a population.

FIG. 3 is a flow diagram of a method for imaging a disease from a sample of a population of subjects with shared characteristics in order to generate a biochemical data image that correspond to a representation of characteristics of a disease associated with the population.

FIG. 4 is a flow diagram showing a method for designing and generating genetically engineered animals in accordance with one embodiment of the present invention.

FIG. 5 is a flowchart illustrating steps that may be followed in accordance with one embodiment of the instant method to derive a relationship between a biochemical analyte image and the corresponding disease associated with a given biochemical analyte image.

FIG. 6 is exemplary biochemical analyte data associated with a given disease (leptin deficient and control mice).

FIG. 7 is an exemplary bar graph representing the relative amount of analytes that are present in five genetically engineered mice.

FIG. 8 is an example of an implementation of the inventive method.

FIG. 9 is a flow chart illustrating the steps that may be followed in accordance with one embodiment of the instant method to design or modify therapeutic treatment of an animal with a disease using a biochemical analyte data image.

FIG. 10 is a flow chart illustrating the steps that may be followed in accordance with one embodiment of the instant method to identify potential pharmaceutical targets of interest using a biochemical analyte data image.

### DETAILED DESCRIPTION

The present invention in one embodiment provides one or more methods of generating and using electronic images comprising biochemical analyte data. A “biochemical analyte data image” (also referred to herein a “biochemical image”) of the present invention is a representation of a plurality of information in a single illustration. That is, a plurality of tests (e.g., measurements of a plurality of analytes) are performed and represented as data from a single test.

The biochemical image may comprise, for example, measurements taken of a plurality of analytes present in a specimen (e.g., blood); one or more measurements taken from different specimens (e.g., blood and urine) from a single subject; or one or more measurements taken from one or more specimens from multiple subjects from a sample of a population. Therefore, an image comprising a plurality of measurements can be used to diagnose and classify a plurality of diseases.

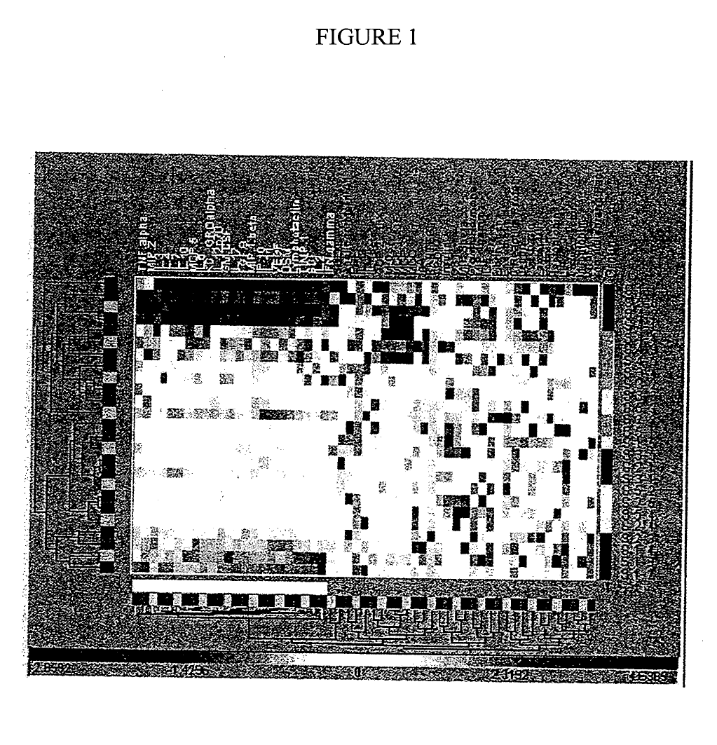

FIG. 1 is an example of a biochemical image of the present invention. For example, numerical data representative of measurements of biochemical analytes in a specimen or within a specimen of a sample from a population is presented as a qualitative image. Each data point of a measurement of an analyte from a specimen is presented in the form of a colored pixel on a computer monitor. In FIG. 1, for example, relatively lower concentrations of a given analyte are presented in shades of blue, while relatively higher concentrations of a given analyte are presented in shades of red. Together a biochemical image is generated from a test subject or subjects in a population sharing a common disease.

A comprehensive database of biochemical images of diseases, states, phenotypes or genotypes can be generated with the invention disclosed herein. Information obtained from a repository of such biochemical images could have applications in drug design and development, genomics research, and disease modeling in animal systems. In some cases, animal disease and ailments could be characterized based on their signature biochemical image, which would have implications in current medical diagnostic and prognostic methods. These and other applications of a database comprising correlations between animal disease and/or a phenotype and biochemical images will become apparent from the description which follows.

In this disclosure, the term “database” will be used interchangeably with “electronic database.” Other terms, which can be equivalently used for “database,” include, but are not limited to, “automated information retrieval system,” “computer readable database,” or “database accessible by a computer.”

Data sets of information may include quantitative and/or qualitative information. Quantitative information may comprise measurements of the concentration of biochemical analytes. Qualitative information may include, but is not limited to, identifiers of the animal subject's disease, for example, its medical history, genotype, and/or phenotype. The term “phenotype” may refer to, for example, genetically engineered animals, including both knock-out and knock-in animals, as well as inbred mice.

In general, the term “analyte” or “biochemical analyte” is meant to be construed broadly and includes “antigens,” “antibodies,” “biochemicals,” “enzymes,” “nucleic acids,” and the like, but is not solely limited to “antigens.” Many types of analytes may be studied, including for example, environmental contaminant analytes, agricultural products, industrial chemicals, water treatment polymers, pharmaceutical drugs, drugs of abuse, and biological analytes, such as antigenic determinants of proteins, polysaccharides, glycoproteins, lipoproteins, nucleic acids, hormones, and parts of organisms, such as viruses, bacteria, fungi, parasites, plants, and microbes.

Quantitative information as to the presence, absence, or relative concentration of analytes present in one or more test samples is referred to herein as “biochemical data,” “biochemical profile,” biochemical “value,” but the terms need not refer to only quantitative information, but are broadly incorporated herein to capture. a wide range of qualitative information from animal subjects that may be of potential interest to medical investigators.

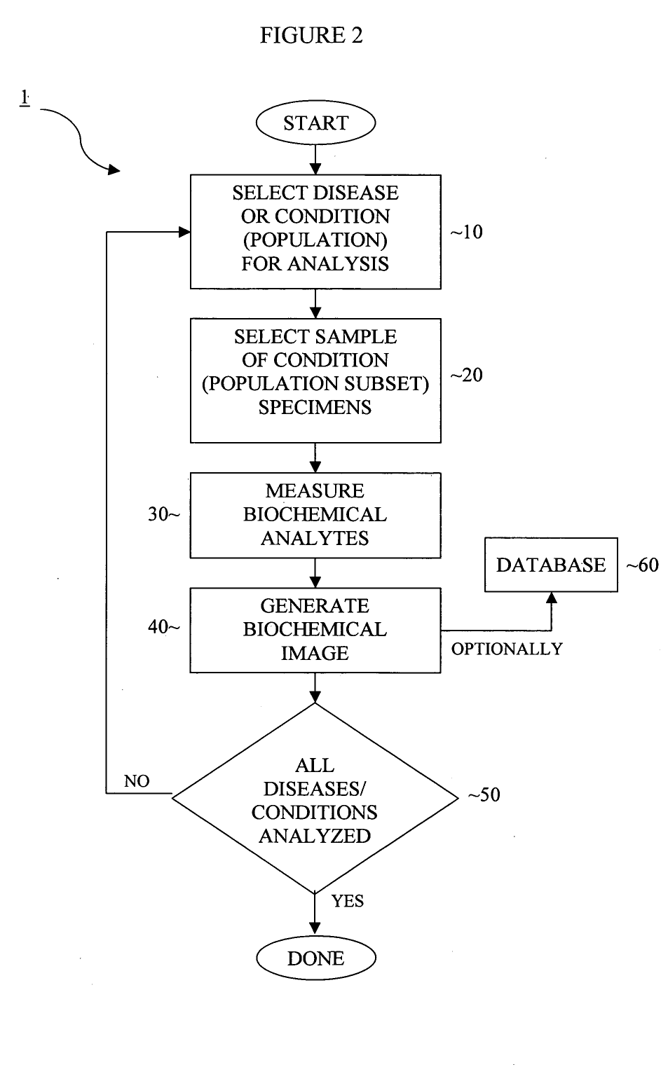

Referring now to FIG. 2, there is a flow diagram of a method 1 for imaging a disease for a given population of animals. “Animals” of the present invention comprise any of living multicellular organisms that may be of potential interest for scientific or medical investigation. Preferably, “animal” refers to vertebrates including, but not limited to humans, primates, rabbits, and rodents, such as, for example, mice, guinea pigs, and rats. The method 1 may be repeated to generate a database of biochemical images of a single disease (e.g., diabetes) from different populations (e.g., teenage children or adults over age 65) and also of different diseases (e.g, diabetes or asthma) in a single population (e.g., teenage children).

In step 10, a disease is selected for analysis. In other words, a population having a common disease or set of characteristics is selected. The disease selected may be studied from the entire population sharing in common the disease, for example, diabetes. Alternatively, the disease selected for study may be further limited to a population having a common age bracket, gender, species, or in the case with humans, race. Thus, for example, the disease selected for analysis may correspond to a population of diabetic patients associated with Caucasian males between ages 35-65 or a population of obese female mice. It should be understood that any population selected for analysis of a disease can correspond to either a control (i.e. “normal”) group or one with a disease (i.e. “abnormal”).

The term “disease” is used to indicate any pathological condition of a living animal or of one of its parts that impairs normal functioning. For example, a “condition” might correspond to a cancer, lung cancer, colon cancer, lymphoma, breast cancer, prostate cancer, or a disease, Alzheimer, Parkinson, diabetes, obesity. A “condition” may also refer to the genotype of an animal (i.e., the genetic background of the subject). Alternatively, “condition” may refer to the phenotype of the animal (i.e., measurable manifestations of a disease or condition in the animal subject).

In step 20, a sample of subjects is selected from the population selected for analysis in step 10. Preferably, the sample includes a number of subjects sufficient to permit a statistically significant analysis of the population as a whole. Thus, preferably, the sample includes a number of subjects such that the biochemical analyte data image generated from the sample corresponds to a statistically significant representation of those biochemical analytes for the population as a whole.

Referring still to FIG. 2, in step 30, a plurality of biochemical analytes are measured from the sample 20. The measurements are representative of exposure of a biological specimens from a sample of subjects of a population to a plurality of biological assays. In generating the biochemical images of the present invention, many types of test specimens from sample 20 can be used. In some embodiments, specimens can comprise biological fluids, mixtures, or preparations thereof. More preferably, one or more test specimens comprise blood samples, mixtures, or preparations thereof. In addition to blood, other bodily fluids may be selected for analysis including, for example, tears, urine, saliva and/or semen.

Exemplary biochemical analytes measured in step 30 include, for example: antigens, antibodies, autoantibodies, peptides, proteins, nucleic acid sequences, enzymes, ions, lipids, drugs, hormones, or combinations thereof. The antigenic analytes, for example, includes bacterial, viral, fungal, mycoplasmal, ridkettsial, chlamydial, and/or protozoal antigens. However, the term “antigen” is understood to include both naturally antigenic species (for example, drugs, proteins, bacteria, bacterial fragments, cells, cell fragments, carbohydrates, nucleic acids, lipids, and viruses, to name a few) and haptens, which may be rendered antigenic under suitable conditions and recognized by antibodies or antibody fragments. Moreover, antigens, for example, include antigens borne by pathogenic agents responsible for a sexually transmitted disease, antigens borne by pathogenic agents responsible for a pulmonary disorder, and/or antigens borne by pathogenic agents responsible for gastrointestinal disorder.

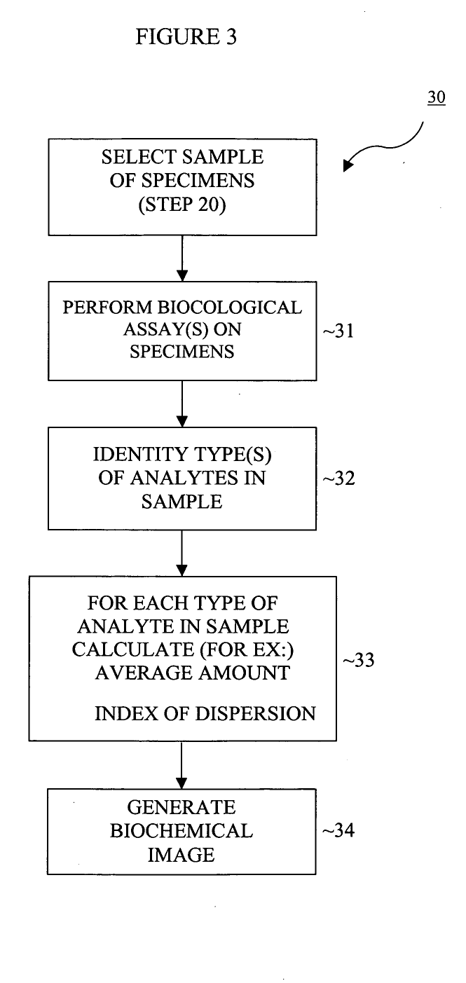

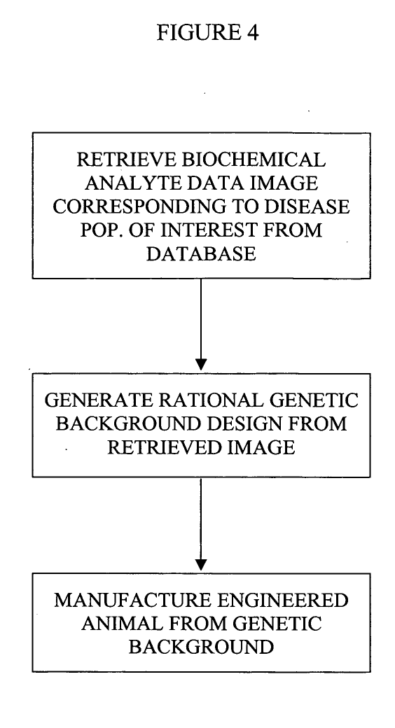

It will be understood by those skilled in the art that biochemical analytes other than those enumerated above may be measured and stored in step 30, and that the use of such other biochemical analytes is within the scope of the present invention. A set of exemplary steps that may be used to measure a sample of specimens and generate the biochemical analyte data enumerated above is shown in detail in FIG. 3 and is discussed more fully below.

In step 40 of FIG. 2, the biochemical data collected in step 30 is electronically processed to generate a biochemical image of the disease. Preferably, in some embodiments, computational software may be used for mining and pooling data from multiple specimens presenting the combined information in a common visual package. An example of such a visual package is presented in FIG. 1 and is available from Omniviz, Inc., of Maynard, Mass. Such software permits the incorporation of relevant information, even from other domains, such as medical history information, or phenotype information, in generating a biochemical image. Once generated, the biochemical images from step 40 may be optionally stored in a database 60 or programmed into a microprocessor to be used for correlations, such as, for example, with images from a test subject.

Referring back to FIG. 2, as indicated in step 50, the imaging process may be repeated for each and any population of interest. All of the biochemical images associated with the population or populations of interest and described above may be stored in the database 60, and may optionally include correlation values as discussed above for each population of interest. By repeating this process for each population of interest, the present invention may be optionally used to generate a database 60 which includes a biochemical analyte data image for many different diseases. Alternatively, a single statistically significant representative image for a given population 20 may be stored electronically or embedded into a software program for comparison or correlation with an image gathered from a test subject.

In this manner, “biochemical imaging” may be used by scientific investigators and/or medical practitioners to gather a biochemical image of a patient and thereby assess the patient's disease based on an image with quantitative and/or qualitative data of the analytes themselves. For example, a specimen from each subject from a sample with a disease may be analyzed and the data may be presented as a biochemical image. The image, rather than the numerical data in this embodiment, may then be compared and correlated with a comprehensive database of biochemical images of disease states to determine the likelihood of a given disease being present.

“Correlations” comprise, for example, comparisons between selected pairs of images. In one example, selected pairs of biochemical images from different populations of cancer patients (e.g., prostate cancer or breast cancer) can be correlated with each other. Such correlations may reveal similarities or differences between cancer types that may aid in the identification and study of a respective disease. Similarly, selected pair of biochemical images from different diabetic populations (e.g., ages 13-18 or ages 55-75) can be correlated with each other, which may reveal information regarding the progression of a disease. It will be understood by those skilled in the art that correlation other than those enumerated above may be made and stored in step 40, and that the use of such other correlations are within the scope of the present invention. For example, selected biochemical images from a diabetic population may be correlated with biochemical images from obese patients.

As mentioned, a biochemical image of a test subject may be correlated with a stored image or a database of stored images. In some embodiments a computer program 65 using one or more biochemical analyte data images 61 may also include a correlation function 62, as illustrated in FIG. 3. A biochemical image 63 from a test subject could be entered into the computer program 65. The program 65 could then correlate the image 63 with one or more images 61 already present in the software or in memory. Once a correlation is made based on user defined parameters, the program 65 can then link the biochemical image 63 with a particular disease. The correlation function 62 is preferably amenable to mathematical or computational manipulation.

This relationship can further provide information relating to the prognosis of a patient. In fact, it is expected that the present invention may enable the detection of disease, such as, for example, cancer, at times earlier than is now possible with conventional technologies, particularly in cases where diseases are manifested in changes in analytes that can be detected by biochemical methods and represented by biochemical imaging. Similarly, the early onset of heart disease and diabetes can be detected in time to allow presymptomatic intervention.

Ultimately, it is an aspect of the present invention to enable the characterization of every disease by a set or panel of biochemical analyte images. Also, particularly where prognosis is desirable, the biochemical image 63 may be gathered and/or correlated with images 61 that have been generated at multiple predetermined times such as, for example, monthly, annually, or over a period of several years to better predict the stage of disease progression in the test subject.

Referring now to FIG. 3, there is shown a flow diagram of the step 30 for imaging the subset 20 of a population of subjects with shared characteristics in order to generate a biochemical analyte image that represents characteristics of a disease associated with the population. In step 31, at least one biochemical assay (preferably, a plurality, and more preferably at least 50) is applied to each specimen from each subject from the sample selected in step 20. The biochemical assay(s) that may be used for a given specimen include, for example, total protein content, total nucleic acid content, total lipid content assays, and/or their respective individual elements such as specific proteins, specific nucleic acid, and specific lipid content assays. In one embodiment, one or more assays are applied to a plurality of specimens in each subject or disease studied.

Preferably, the plurality of biochemical assays are performed in a single experiment using. For example, in some embodiments, analytical reagents are coupled to microspheres which are then analyzed in a flow cytometer. This technology allows the simultaneous determination of the concentration and identity of multiple biochemicals in a single sample of blood or other biological fluid and is described in U.S. Pat. No. 6,592,822, the disclosure of which is incorporated by reference herein.

Preferred reagents bound to the microspheres may comprise a small molecule, natural product, synthetic polymer, peptide, polypeptide, polysaccharide, lipid, nucleic acid, or combination thereof. In performing the methods of the present invention, it may be useful to add one or more supplemental reagents to assist, enhance, or facilitate the generation of biochemical data. Such supplemental reagents may comprise a substrate, antibody, affinity reagent, label, or combinations thereof. One of ordinary skill in the art may also find that there is some advantage to performing certain additional steps. For example, one might choose to further filter the exposed microspheres from the one or more specimens prior to passing the filtered microspheres through the flow analyzer.

The molecular interactions between reagent and analyte may be optimized for both sensitivity and specificity. Preferred analyte:reagent (or vice-versa) couples, however, include, but are not limited to, antigen:specifc immunoglobulins; hormone:hormone receptor; nucleic acid strand:complementary polynucleotide strand; avidin:biotin; protein A:immunoglobulin; protein G:IgG immunoglobulins; enzyme:substrate; lectin:specific carbohydrate; drug:protein; small molecule:protein, and the like.

It will be understood by one of ordinary skill in the art that the assays may alternatively comprise any biological assay or reagent known and available or that may become available to one of ordinary skill in the art. These assays and reagents include, but are not limited to, conventional blood counts (CBC), Western blots, Northern blots, Southern blots, polymerase chain reaction (PCR) analysis, restriction mappings, DNA footprintings, nucleic acid arrays, enzyme-linked immunosorbent assays (ELISA), Bradford assays, BCA assays, single and 2D electrophoresis and staining, enzymatic assays, and spectroscopy.

Referring back to FIG. 3, in step 32, the biochemical data from step 31 is analyzed in order to identify types of biochemical analytes that are present in the sample. The types of analytes identified for analysis preferably correspond to the types of analytes that distinguish the disease population of interest from other control populations. For example, where diseases of the immune system are known in the sample of the population, cytokines may be particularly examined. In step 33, three exemplary values are preferably determined for each type of analyte that was identified in step 32. More particularly, for each identified type of analyte, the following values are determined in step 33: (i) the average amount of the particular type of analyte in the sample, (ii) an index of dispersion associated with the measured average amount of the particular type of analyte, and (iii) the p-value associated with the measurement.

Referring still to step 33, for each identified type of analyte, the average amount of the particular type of analyte in the sample of the population and the index of dispersion associated with the measured average amount of the particular type of analyte are determined by first analyzing the biochemical assay information corresponding to each sample of the population in order to determine the average amount of the particular type of analyte in each such specimen. By performing such an analysis on each specimen in the sample, a distribution of analyte values for the particular type of analyte may then be obtained.

An average amount index representative of an average amount of the particular type of analyte in the population is then calculated by taking the statistical average of this distribution. Similarly, a standard deviation about the average amount of the particular type of analyte in the population is calculated by, for example, taking the standard deviation, standard error, or standard error of the mean of the distribution of analyte amount values obtained for the particular type of analyte from the sample.

A p-value is a measure of how much evidence can be weighted against the null hypotheses (i.e., a hypothesis that presumes no change or no effect of a treatment). The p-value measures consistency by calculating the probability of observing the results from your sample of data or a sample with results more extreme, assuming the null hypothesis is true. The smaller the p-value, the greater the inconsistency.

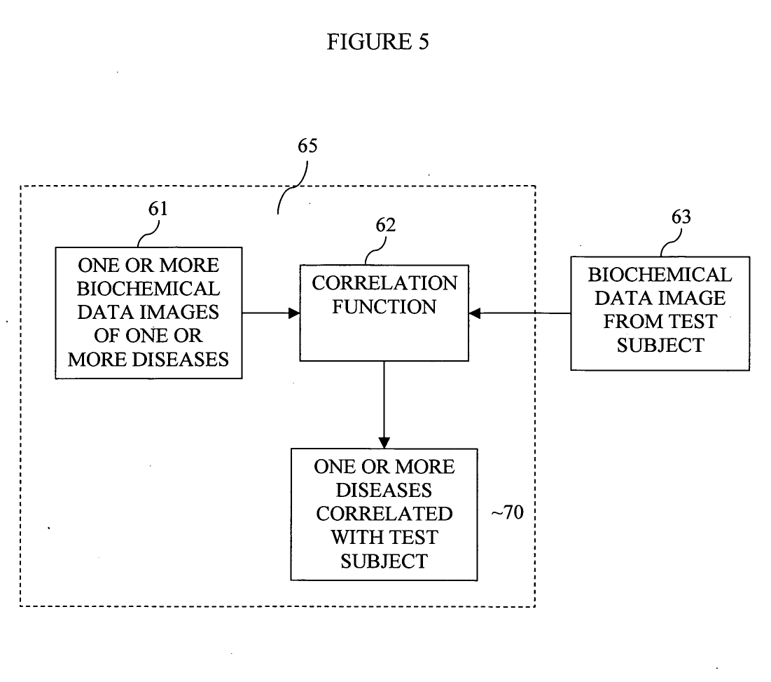

The biochemical images associated with each disease studied may also be processed to collectively represent a “blueprint” of the disease in the population 20 and may be used, inter alia to rationally design and then manufacture animal models corresponding to the diseased population. For example, as depicted in a flow chart in FIG. 5, a model designed for a given disease may include animals that have been genetically engineered to include and/or exclude genes and protein factors that yield an animal with a biochemical analyte data profile similar to that observed in the human disease population. Thus, in one particular example, the leptin deficient mice may be generated to reflect leptin deficiency commonly associated with obesity in mammals. Alternatively, biochemical images taken from animals genetically engineered to mimic a human disease, may also be used for comparison with biochemical images of humans with the respective disease. In this manner, biochemical images of a disease may be used to validate the use of an animal model to study the disease.

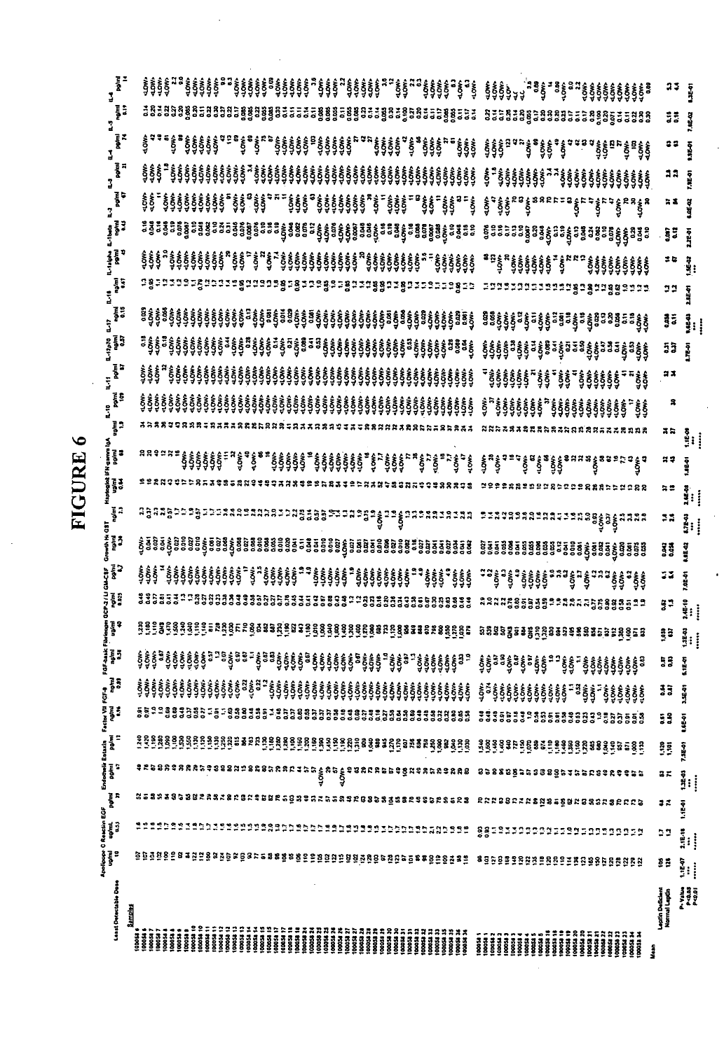

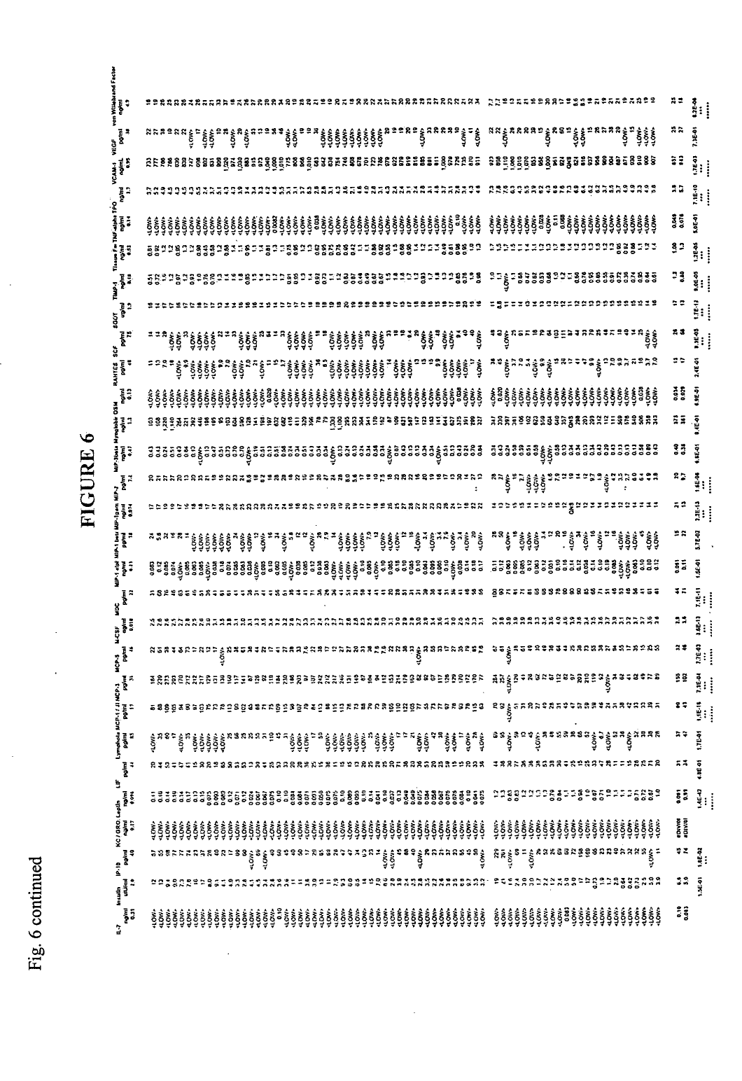

FIG. 6 is one example of a biochemical analyte data that can be used in the generation of a biochemical image of the instant invention. The data comprises measurements of 57 analytes listed across the top of the figure. Two populations of mice were studied—obese mice and control mice. From this population, 24 obese mice and 12 control mice were sampled. In this particular example, the obese mice were genetically engineered by ablating the leptin gene.

A blood specimen was obtained from each of the mice and each blood specimen was assayed in two independent experiments for the presence and concentration of analytes. Microsphere coupled reagents were incubated with the blood specimen and analyzed by flow cytometry. The reading for each analyte in each experiment is listed in the table. The mean reading for each analyte in each sample population is also listed across the bottom of the figure. In addition, for each analyte, the corresponding p-value is shown.

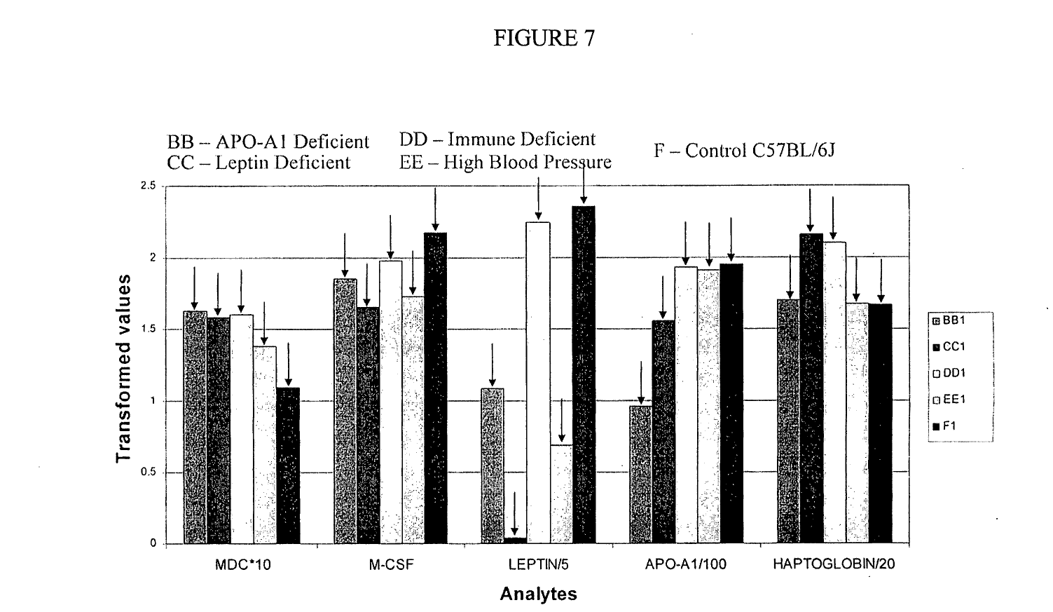

Referring now to. FIG. 7, in an independent experiment, a data profile of 75 analytes similar to the profile in FIG. 6 was generated for five populations of mice—apoprotein-deficient, leptin-deficient, immuno-compromised, exhibiting high-blood pressure, and control. Less than 1 ml of blood was drawn from each animal. Sixteen to eighteen mice were sampled for each population. The data was then subjected to a computer implemented algorithm to determine the least number of analytes that would be necessary to distinguish the populations based on the biochemical analyte data alone. The algorithm selected five analytes as being sufficient—MDC*10, M-CSF, Leptin/5, Apo-A1/100, and Haptoglobin/20. The relative amount of each of analytes that were present in the five genetically engineered mice populations is presented in FIG. 7.

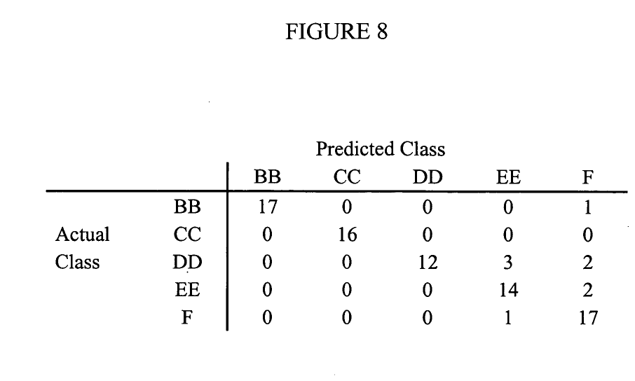

FIG. 8 is a table representing the accuracy in predicting the population (i.e., disease) affecting individual mice based on the five analytes selected above in this experiment. As is shown, apoprotein-deficient and control mice were correctly identified 17 of 18 times (94.4%), all of 16 leptin mice were correctly identified (100%), the immunocompromised mice were correctly identified 12 of 17 times (70.6%), and mice with high blood pressure were correctly identified 14 of 16 times (87.5%). Therefore, in one embodiment of the present invention, measurements from five or more analytes may be used in the generation of a biochemical image and may be sufficient to identify a disease.

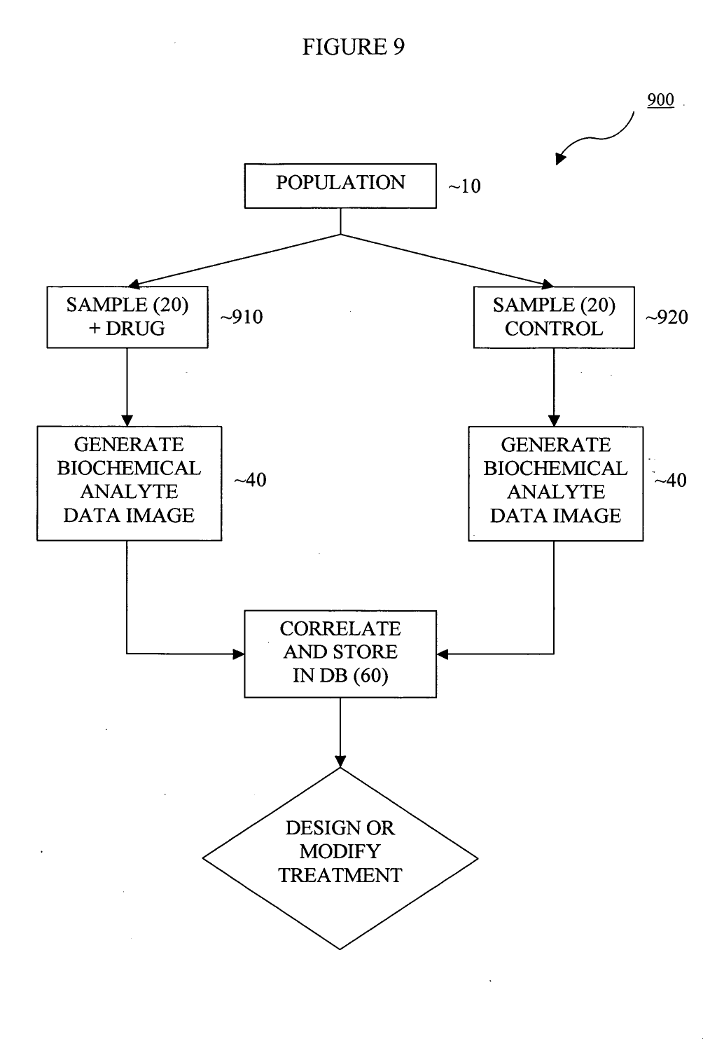

It will be clear to one of ordinary skill in the art from the teachings disclosed herein the many applications of a database comprising biochemical analyte data images from a plurality of disease, genotypes, or phenotypes. For example, the use of many drugs have undesirable side effects. Often times the underlying biochemical basis for the side effect is unknown or poorly understood. FIG. 9 is a flow chart illustrating the steps 900 that may be followed in accordance with one embodiment of the instant method to improve drug safety and efficacy or therapeutic treatment of an animal with a disease based on a biochemical analyte profile.

Using the instant inventive method, a sample of a population sharing a common disease could be divided into two subpopulations 910, 920, one treated with a drug of interest and one without. Biological specimens, preferably blood and preferably from a statistically representative sample size, could be donated and analyzed of its biochemical analytes. A biochemical analyte data image 40 can then be generated from the data gathered from each of the sample populations 910 and 920. The information may be analyzed for differences in specific analytes or analyte groups in the two subpopulations. Such differences may be representative of biochemical manifestations of drug safety concerns, drug efficacy, and generally, drug side effects. Based on the differences in analyte images between subpopulations 910, 920, a new or modified treatment may be developed to counter some or all of the side effects.and improve drug performance and efficacy.

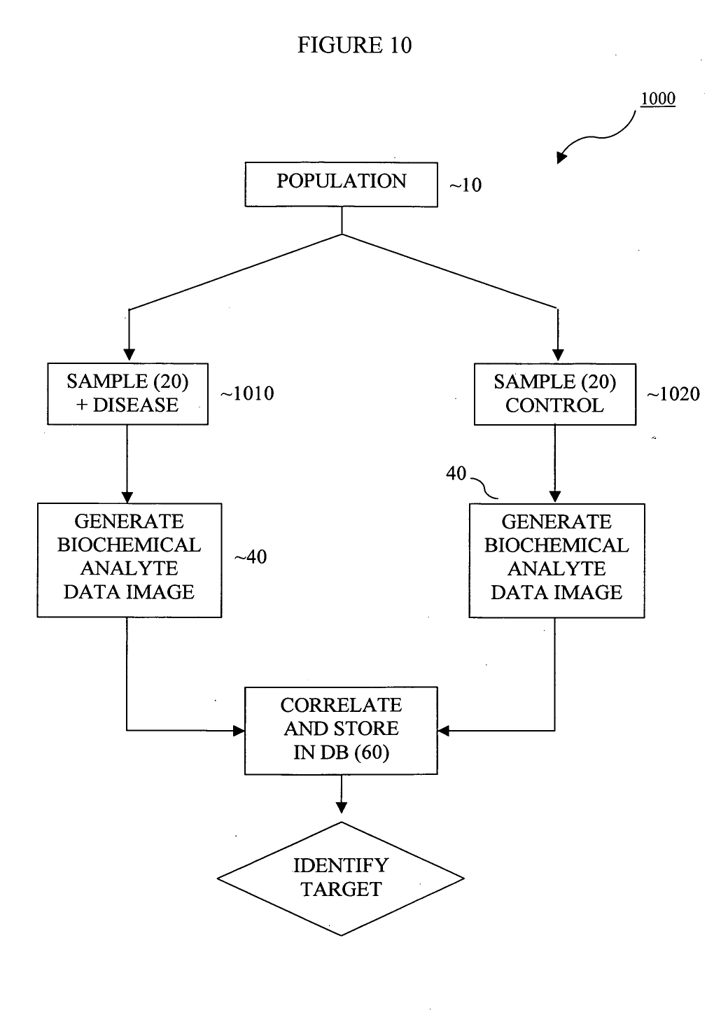

In another similar example, the teachings of the present invention may be used for identifying targets for therapeutic intervention. FIG. 10 is a flow chart illustrating the steps 1000 that may be followed in accordance with one embodiment of the instant method to identify pharmaceutical targets for therapeutic treatment of an animal with a disease based on a biochemical analyte profile.

Using the instant inventive method, a sample of a population could be divided into two subpopulations 1010, 1020, one sharing a common disease and one without, respectively. Biological specimens, preferably blood and preferably from a statistically representative sample size, could be donated and analyzed of its biochemical analytes. A biochemical analyte data image can then be generated from the data gathered from each of the sample subpopulations 1010, 1020. The information may be analyzed for differences in specific analytes or analyte groups in the two subpopulations. Such differences may be representative of specific manifestations of the disease that can distinguish the two groups on a biochemical level. Based on the differences then, a new or modified treatment may be developed to cure, alleviate, or generally, treat the biochemical differences between the two subpopulations.

The method of the present invention can be used to generate a biochemical analyte data images and optionally the correlation values discussed above for a disease including, but not limited to, neoplastic, neurodegenerative, skeletal, muscular, connective tissue, skin, organ, metabolic, addictive, psychiatric disease, or combinations thereof.

The many features and advantages of the invention are apparent from the detailed specification, and thus, it is intended by the appended claims to cover at least one or more such features and advantages of the invention. Further, since numerous modifications and variations will readily occur to those skilled in the art based on the teachings herein, it is not desired to limit the invention to the exact construction and operation illustrated and described, and accordingly, all suitable modifications and equivalents may be considered to be covered by the appended claims.

1. A method for generating a biochemical image of a disease state comprising:
(a) obtaining one or more specimens for a disease state from a sample population of subjects with a disease,
(b) assaying each of the specimens for concentration values of a plurality of biochemical analytes,
(c) determining for each disease a distribution of said concentration values for each biochemical analyte,
(d) calculating average values for said concentration values for each biochemical analyte,
(e) storing the distribution and average values in a database, and
(f) generating a biochemical image representing the values from (d).

2. The method of claim 1, wherein the specimens are derived from normal subjects.

3. The method of claim 1, wherein the specimens are derived from subjects comprising any disease characterized by neoplasia, neurodegeneration, or immunodeficiency.

4. The method of claim 1, wherein the specimens are derived from each of a normal population and an abnormal population, and wherein in the determination of distribution of values and corresponding values, data from the normal population is used to determine a distribution of values and corresponding values for normal subjects and data from abnormal subjects is used to determine a distribution of values and corresponding indices for abnormal subjects.

5. The method of claim 1, wherein the assaying comprises microspheres analyzed in a flow cytometer.

6. A method for identifying a genotype from a biochemical phenotype comprising:
(a) providing one or more test specimens from a subset of a population of subjects with shared a shared genotype,
(b) assaying for each of the test specimens for the concentration values of a plurality of biochemical analytes,
(c) determining for each genotype from the assaying in (b) a distribution of values for each biochemical analyte,
(d) calculating average values for each of the distribution of values in (c),
(e) deriving for each genotype from the assaying in (b) a mathematical correlation between the biochemical phenotype obtained from the values calculated in (d) and the genotype,
(f) generating a biochemical image comprising the correlation data,
(g) providing to the user access to said average values and correlation data in said database, and
wherein the number of specimens in (a) includes a sufficient number of specimens such that the values correspond to a statistically significant representation of those values for the population as a whole.

7. A method for identifying a disease from a biochemical phenotype comprising:
(a) providing one or more test samples derived from a test subject;
(b) exposing the one or more test samples to a panel of biochemical assays to gather values for a plurality of biochemical analytes;
(c) generating a biochemical image representing the values of (b);
(d) comparing the biochemical analyte image generated from the one or more test samples from the test subject with a database of accumulated biochemical analyte image from test samples taken from a plurality of diseases, which accumulated data provides a relationship between one or more predetermined biochemical images and the disease of a plurality of subjects whose accumulated biochemical analyte data share similar features; and
(e) identifying a disease in the test subject based, at least in part, on the results of the comparison.

8. A method according to claim 7, wherein the disease is a genotype.

9. A method according to claim 7, wherein the disease is a disease.

10. A method of generating an animal model of a disease from a biochemical phenotype of the disease comprising:
(a) obtaining one or more test specimens from a population of subjects with a shared disease;
(b) exposing the one or more test samples to a plurality of biochemical assays to gather values for a plurality of biochemical analyte data;
(c) determining a relationship between one or more biochemical analyte data images and the disease of the population of subjects whose accumulated biochemical indices share similar features; and
(d) genetically manipulating an animal having to comprise one or more biochemical analyte data image associated with the disease of the population of subjects.

11. A method of generating an animal model of a genotype from a biochemical phenotype of the disease comprising:
(a) obtaining one or more test specimens from a population of subjects with a shared genotype;
(b) exposing the one or more test samples to a plurality of biochemical assays to gather values for a plurality of biochemical analyte data;
(c) generating a biochemical image representing the values from (b);
(d) determining a relationship between one or more biochemical analyte data and the genotype of the population of subjects whose accumulated biochemical analyte data share similar features; and
(e) genetically manipulating an animal having one or more biochemical indices associated with the genotype of the population of subjects.

12. A computer implemented method for providing information on a disease to a user comprising:
(a) obtaining one or more test specimens for a plurality of diseases from a subset of a population of subjects with a shared disease,
(b) assaying each of the test specimens for the concentration values of a plurality of biochemical analytes,
(c) determining for each disease from the assaying in (b) a distribution of values for each biochemical analyte,
(d) calculating average values for each of the distribution of values in (c),
(e) generating a biochemical image representing the values from (d), and
(f) providing to the user access to said distributing and average values in said database.
